(19) United States
(12) Patent Application Publication
(10) Pub. No.: US 2010/0010527 A1
(43) Pub. Date: Jan. 14, 2010

(54) Safety lancet

(76) Inventor: Gary Ge Chen, Anaheim, CA (US)

Correspondence Address: BRENT A. CAPEHART, 1413 MCCOY DRIVE, SUITE A, HARRISON, AR, 72601, US

(21) Appl. No.: 12/171,486

(22) Filed: Jul. 11, 2008

(51) Int. Cl. A61B 17/32

(52) U.S. Cl. 606/181

(57) ABSTRACT

The present invention is directed toward a safety lancet having a safety cap slidably engaged with a hub having a cannula such that in a pre-use position the safety cap allows access to the cannula and in post-use position, the safety cap prevents access to the cannula.

### REFERENCE TO PENDING APPLICATIONS

This application is not based on any pending domestic or international patent application.

### BACKGROUND OF THE INVENTION

The present invention is generally directed toward a safety lancet, more specifically, toward an safety lancet having safety cap that will eliminate any chance of it being exposed and accidentally cause an injury in an un-intended application.

Diabetics must test for stat glucose levels up in order to determine whether or not they should administer insulin. The procedure involves drawing a drop of blood from the diabetic's finger, placing the drop of blood on a commercially available blood glucose test strip, and comparing the resulting color of the test strip with a standard chart to determine the blood glucose concentration.

In order to draw the need drop of blood, a lancet, a medical device with a very sharp and tiny pointed instrument, is used. Diabetic patients use these devices to prick on the tip of a finger to produce a very small amount of blood which can then be collected and tested for its sugar content. The prior art lancets have to be handled very carefully by patient or person. These lancets are exposed and can be dangerous to accidental pricks and injury. Further, after use, they have to be disposed in a specially designed collection container or risk exposure to cross-contamination to another person. Clearly, there is a need for an improved lancet.

### BRIEF SUMMARY OF THE INVENTION

The present invention satisfies the needs discussed above. The present invention is generally directed toward a safety lancet, more specifically, toward a safety lancet having safety cap that will eliminate any chance of it being exposed and accidentally cause an injury in an un-intended application.

One aspect of the present invention discloses a hub assembly secured within a pen body. The hub assembly has a hub body and a series of grooves. A safety cap assembly is slidably connected to the hub assembly, such that is engages the different grooves depending on whether it is in a pre-use position or a post-use position. In the pre-use position, a cannula that is located on the hub assembly is exposed and is ready for use. In a post-use position, the cannula is cover, or otherwise shielded, from contact.

An adjustment cap is also disclosed. This adjustment cap allows for the cannula to extend through an opening therein. Once the blood has been drawn, the adjustment cap is removed. In so doing, it engages with the safety cap pulling it from a pre-use position to its post-use position.

Upon reading the above description, various alternative embodiments will become obvious to those skilled in the art. These embodiments are to be considered within the scope and spirit of the subject invention, which is only to be limited by the claims which follow and their equivalents.

### BRIEF DESCRIPTION OF THE DRAWINGS

FIG. 1 is a prospective, cut-away view of an embodiment of the present invention

FIG. 2 is a cut-away view of an embodiment of the hub assembly of the present invention.

FIG. 3 is a side view of the embodiment of the FIG. 1 after use of an embodiment of the hub assembly of FIG. 2.

FIGS. 4-6 are additional embodiments of the safety cap assembly of the present invention.

### DETAILED DESCRIPTION OF THE INVENTION

The attached drawing demonstrates an embodiment of the present invention. It is to be understood that the invention is not limited in its application to the details of the construction and arrangement of parts illustrated in the accompanying drawings. The invention is capable of other embodiments and of being practiced or carried out in a variety of ways. It is to be understood that the phraseology and terminology employed herein are for the purpose of description and not of limitation.

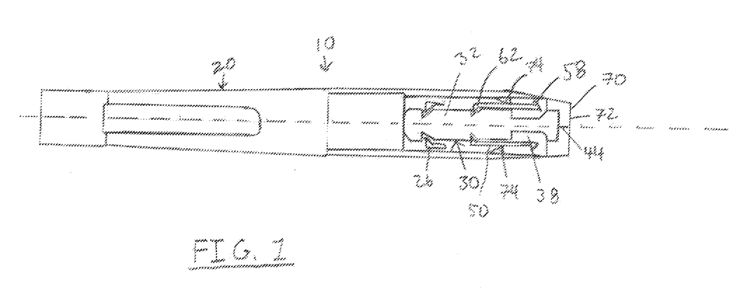

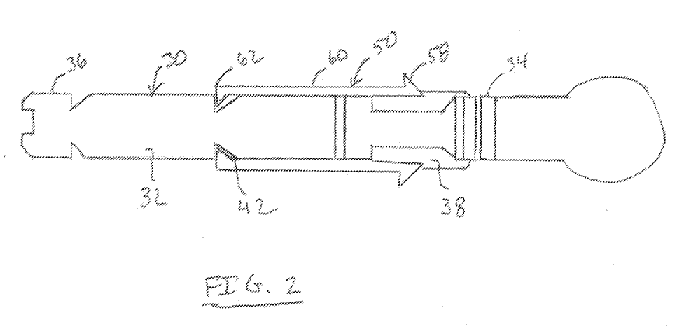

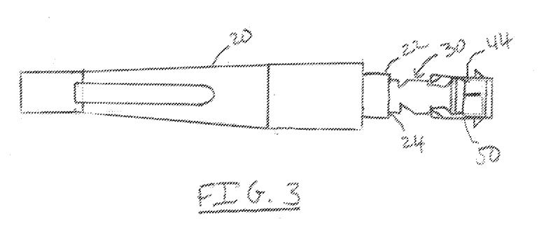

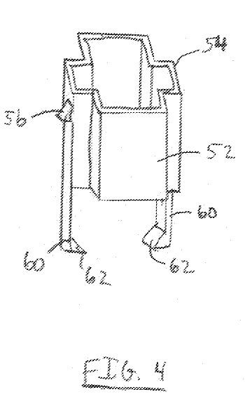

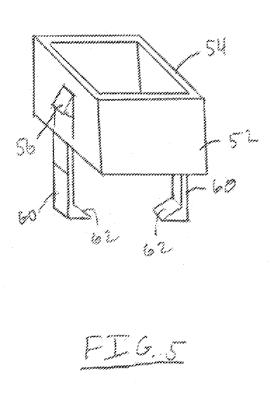

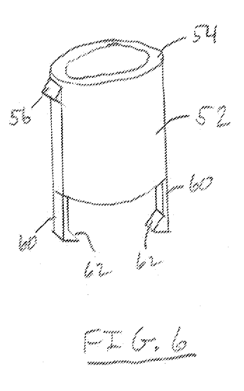

As shown in FIG. 1, an embodiment 10 of the present invention has a pen body assembly 20 having a hub end 22 having an opening 24. A hub assembly 30 secured within the pen body opening 24. The hub assembly 30 has a hub body 32, a cannula 44 and a safety cap assembly 50.

The hub body 32 has a cannula end 34 and a pen body end 36. Further, the hub body 32 has a series of grooves, a cannula end groove 38 located proximate to the cannula end 34, a pen body end groove 40 located proximate to the pen body end 36 and an intermediate groove 42 located between the cannula end groove 38 and the pen body end groove 40.

A cannula 44 is secured to the cannula end 34 and extends away therefrom.

The safety cap assembly 50 is dimensioned such that is can slide along the outside of the hub body 32. The safety cap assembly is shown as cylindrical in dimension. This is illustrative and other dimensions, such rectangular, can be used. The safety cap assembly 50 has a safety cap body 52 having an upper end 54 and a lower end 56. A plurality of safety cap pulling hooks 58 are located near, or proximate, to the upper end 54 and extending outward. A plurality of prongs 60 extend away from the lower end 56. Each of the plurality of prongs 60 have an anti-back hook 62 extend inward.

The anti-back hooks 62 are dimensioned to engage with the intermediate groove 42 when the safety cap assembly 50 is a pre-use position and to engage with the cannula end groove 38 when the safety cap assembly 50 is in a post-use position.

An adjustment cap 70 engages with the hub assembly 30 by being dimensioned to fit over the hub assembly 30 and allow a portion of the cannula to 44 to extend beyond the adjustment cap 70 through a cannula opening 72. Further, the adjustment cap 70 has a plurality of adjustment cap pulling hooks 74 located its inter surface. These hooks 74 engage the safety cap pulling hooks 58 such that when the adjustment cap 70 is removed, the safety cap assembly 50 is moved from its pre-use position to its post-use position allowing safety cap body 52 to shield the cannula 44.

Another embodiment of the present invention allows for the hub assembly 30 to be removably secured to the pen body assembly 20. In this embodiment, the pen body includes a plurality of locking hooks 26 that engage with the pen body end groove 40 located within the hub body 32.

The operation of the embodiment of the present invention involves assembling the safety cap assembly 50 onto the hub assembly 30 by sliding the safety cap body 52 over the hub body 32 until the anti-back hooks 62 engage with the intermediate groove 42. The pen body end 36 of the hub assembly 30 is then secured within said pen body 20. In one embodiment, locking hooks 26 within the pen body 20 engage with the pen body end groove 40 locking in hub assembly 30 in position.

A needle cap 48 is removed exposing the cannula 44 and is replaced with the adjustment cap 70. The adjustment cap 70 is positioned such that the desired length of the cannula 44 extends through the cannula opening 72 located within the adjustment cap 70.

Once the blood has been collected, the adjustment cap 70 is removed by pulling it away from the pen body 20. The action of removing the adjustment cap 70 allows the adjustment cap pulling hooks 74 engage with the safety cap pulling hooks 58. Due to the different resistance caused by the different angle and thickness of the various contact fields, the resistance of the adjustment cap pulling hooks 74 and safety cap pulling hooks 58 are greater than that of the anti-back hooks 62 and intermediate groove 42 but less than that of the pen body locking hooks 26 that are engaged with the pen body end groove 40. This causes the anti-back hooks 62 to disengage from said intermediate groove allowing the safety cap body to slide along the hub body until it engages with the cannula end groove 38. When this occurs, the adjustment cap pulling hooks 74 disengage from the safety cap pulling hooks 58 allowing the adjustment cap 70 to be released. The safety cap body 52 now extends beyond the cannula end 34 of the hub assembly 30, essentially covering, or otherwise shielding the cannula 44. Thus preventing any accidental contact.

While the invention has been described with a certain degree of particularity, it is manifest that many changes may be made in the details of construction and the arrangement of components without departing from the spirit and scope of this disclosure. It is understood that the invention is not limited to the embodiments set forth herein for purposes of exemplification.

1. A safety lancet comprising:
a pen body assembly having a hub end, said hub end having an opening;
a hub assembly secured within said opening of said hub end of said pen body, said hub assembly having a hub body, a cannula and a safety cap assembly,
said hub body having a cannula end and a pen body end, said hub body end having a cannula end groove proximate said cannula end, a pen body end groove proximate said pen body end and an intermediate groove located between said cannula end groove and said pen body end groove,
said cannula secured to said cannula end and extending away therefrom,
said safety cap assembly being dimensioned to slideably engage said hub body having a safety cap body having an upper end and a lower end, a plurality of safety cap pulling hooks located proximate to said upper end and extending outward, a plurality of prongs extending away from said lower end, each of said plurality of prongs having an anti-back hook extending inward,
wherein each of said anti-back hooks being dimensioned to engage said intermediate groove when said safety cap assembly is in a pre-use position and to engage said cannula groove when said safety cap assembly is in a post-use position;
an adjustment cap assembly having a adjustment cap dimensioned to engage said hub assembly, said adjustment cap having a plurality of adjustment cap pulling hooks located along the inter surface of said adjustment cap dimensioned to engage said safety cap pulling hooks, and a cannula opening to allow access for said cannula,
wherein said adjustment cap pulling hooks engage said safety cap pulling hooks such that when said adjustment cap assembly is removed, said safety cap assembly is moved from its pre-use position to its post-use position allowing said safety cap body to shield said cannula.

2. The safety lancet of claim 1 wherein said hub assembly is removeably secured to said pen body assembly.

3. The safety lancet of claim 2 wherein said pen body further comprises a plurality of securing hooks to securely engage said pen body grooves located on said hub body.

4. The safety lancet of claim 1 wherein said safety cap body is cylindrical in dimension.

5. The safety lancet of claim 2 wherein said safety cap body is rectangular in dimension.
